SEQUENCE LISTING (51 sequences)

SEQ ID NO: 1 - DNA, 1823 bp, Homo sapiens
  ref <308>: DATABASE ACCESSION NUMBER: NCBI/AF013171
  ref <309>: DATABASE ENTRY DATE: 1997-09-19
  ref <313>: RELEVANT RESIDUES: (1)..(1823)
  ref <308>: DATABASE ACCESSION NUMBER: NCBI/NM_033012.2
  ref <309>: DATABASE ENTRY DATE: 2002-07-31
  ref <313>: RELEVANT RESIDUES: (1)..(1823)
  ref <308>: DATABASE ACCESSION NUMBER: NCBI/AF053712.1
  ref <309>: DATABASE ENTRY DATE: 1998-05-09
  ref <313>: RELEVANT RESIDUES: (1)..(1823)
  ref <308>: DATABASE ACCESSION NUMBER: NCBI/AF019047.1
  ref <309>: DATABASE ENTRY DATE: 1997-11-22
  ref <313>: RELEVANT RESIDUES: (1)..(1823)
cagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcgggggtggggtaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggcccaacggtacacgactcagtatccatgctcttgaccttgtagagaacacgcgtatttacagccagtgggagatgttagactcatggtgtgttacacaatggtttttaaattttgtaatgaattcctagaattaaaccagattggagcaattacgggttgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatctgtcaagagtagttatataattgttgaacaggtgtttttccacaagtgccgcaaattgtacctttttttgtttttttcaaaatagaaaagttattagtggtttatcagcaaaaaagtccaattttaatttagtaaatgttatcttatactgtacaataaaaacattgcctttgaatgttaattttttggtacaaaagtcgacggccgc

SEQ ID NO: 2 - DNA, 1769 bp, Homo sapiens
  ref <308>: DATABASE ACCESSION NUMBER: NCBI/NM_003810.2
  ref <309>: DATABASE ENTRY DATE: 2002-10-07
  ref <313>: RELEVANT RESIDUES: (1)..(1769)
  ref <308>: DATABASE ACCESSION NUMBER: NCBI/U57059.1
  ref <309>: DATABASE ENTRY DATE: 1999-03-04
  ref <313>: RELEVANT RESIDUES: (1)..(1769)
  ref <308>: DATABASE ACCESSION NUMBER: NCBI/U37518.1
  ref <309>: DATABASE ENTRY DATE: 1996-01-06
  ref <313>: RELEVANT RESIDUES: (1)..(1769)
  ref <308>: DATABASE ACCESSION NUMBER: NCBI/BC032722.1
  ref <309>: DATABASE ENTRY DATE: 2002-06-27
  ref <313>: RELEVANT RESIDUES: (1)..(1769)
cctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagaaaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagcgcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtgcaaaaattagctgggtgtgttggcacatgcctgtagtcccagctacttgagaggctgaggcaggagaatcgtttgaacccgggaggcagaggttgcagtgtggtgagatcatgccactacactccagcctggcgacagagcgagacttggtttcaaaaaaaaaaaaaaaaaaaaacttcagtaagtacgtgttatttttttcaataaaattctattacagtatgtcaaaaaaaaaaaaaaaaaa

SEQ ID NO: 3 - DNA, 1803 bp, Homo sapiens
  ref <308>: DATABASE ACCESSION NUMBER: NCBI/X67878.1
  ref <309>: DATABASE ENTRY DATE: 1997-06-06
  ref <313>: RELEVANT RESIDUES: (1)..(1803)
  ref <308>: DATABASE ACCESSION NUMBER: NCBI/NM_000074.1
  ref <309>: DATABASE ENTRY DATE: 2002-04-10
  ref <313>: RELEVANT RESIDUES: (1)..(1803)
  ref <308>: DATABASE ACCESSION NUMBER: NCBI/L07414.1
  ref <309>: DATABASE ENTRY DATE: 1993-04-27
  ref <313>: RELEVANT RESIDUES: (1)..(1803)
tgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacatt

SEQ ID NO: 4 - DNA, 1643 bp, Homo sapiens
gcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaattgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgctt

SEQ ID NO: 5 - DNA, 1325 bp, Homo sapiens
gaggtttattgggcctcggtcctcctgcacctgctgcctggatccccggcctgcctgggcctgggccttggttctccccatgacaccacctgaacgtctcttcctcccaagggtgtgtggcaccaccctacacctcctccttctggggctgctgctggttctgctgcctggggcccaggggctccctggtgttggcctcacaccttcagctgcccagactgcccgtcagcaccccaagatgcatcttgcccacagcaccctcaaacctgctgctcacctcattggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccacctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctgtagaacttggaaaaatccagaaagaaaaaataattgatttcaagaccttctccccattctgcctccattctgaccatttcaggggtcgtcaccacctctcctttggccattccaacagctcaagtcttccctgatcaagtcaccggagctttcaaagaaggaattctaggcatcccaggggacccacactccctgaaccatccctgatgtctgtctggctgaggatttcaagcctgcctaggaattcccagcccaaagctgttggtcttgtccaccagctaggtggggcctagatccacacacagaggaagagcaggcacatggaggagcttgggggatgactagaggcagggaggggactatttatgaaggcaaaaaaattaaattatttatttatggaggatggagagagggaataatagaagaacatccaaggagaaacagagacaggcccaagagatgaagagtgagagggcatgcgcacaaggctgaccaagagagaaagaagtaggcatgagggatcacagggccccagaaggcagggaaaggctctgaaagccagctgccgaccagagccccacacggaggcatctgcaccctcgatgaagcccaataaacctcttttctctga

SEQ ID NO: 6 - DNA, 5307 bp, Homo sapiens
  misc_feature (4242)..(4242): n = a, t, c or g
  misc_feature (4471)..(4471): n = a, t, c or g
  misc_feature (4523)..(4523): n = a, t, c or g
  misc_feature (4529)..(4529): n = a, t, c or g
  misc_feature (4531)..(4531): n = a, t, c or g
  misc_feature (4545)..(4545): n = a, t, c or g
attccctcggcgggccgagcctcccctctctcccgcccctcctcctccctttcccacccctcggagtagagctgcacatgcggctgctccctgctccgtcccgcccagccactgtcgcgcaggaacgggtccctgcagcccccagccgatggcaggacagtagccgcctgtcagaggtcgtgaacggctgaggcagacgcagcggctcccgggcctcaagagagtggatgtctccggaggccatgggctacccggaggtggagcgcagggaactcctgcctgcagcagcgccgcgggagcgagggagccagggctgcgggtgtggcggggcccctgcccgggcgggcgaagggaacagctgcctgctcttcctgggtttctttggcctctcgctggccctccacctgctgacgttgtgctgctacctagagttgcgctcggagttgcggcgggaacgtggagccgagtcccgccttggcggctcgggcacccctggcacctctggcaccctaagcagcctcggtggcctcgaccctgacagccccatcaccagtcaccttgggcagccgtcacctaagcagcagccattggaaccgggagaagccgcactccactctgactcccaggacgggcaccagatggccctattgaatttcttcttccctgatgaaaagccatactctgaagaagaaagtaggcgtgttcgccgcaataaaagaagcaaaagcaatgaaggagcagatggcccagttaaaaacaagaaaaagggaaagaaagcaggacctcctggacccaatggccctccaggacccccaggacctccaggaccccagggacccccaggaattccagggattcctggaattccaggaacaactgttatgggaccacctggtcctccaggtcctcctggtcctcaaggaccccctggcctccagggaccttctggtgctgctgataaagctggaactcgagaaaaccagccagctgtggtgcatctacagggccaagggtcagcaattcaagtcaagaatgatctttcaggtggagtgctcaatgactggtctcgcatcactatgaaccccaaggtgtttaagctacatccccgcagcggggagctggaggtactggtggacggcacctacttcatctatagtcaggtagaagtatactacatcaacttcactgactttgccagctatgaggtggtggtggatgagaagcccttcctgcagtgcacacgcagcatcgagacgggcaagaccaactacaacacttgctataccgcaggcgtctgcctcctcaaggcccggcagaagatcgccgtcaagatggtgcacgctgacatctccatcaacatgagcaagcacaccacgttctttggggccatcaggctgggtgaagcccctgcatcctagattccccccattttgcctctgtccgtgccccttccctgggtttgggagccaggactcccagaacctctaagtgctgctgtggagtgaggtgtattggtgttgcagccgcagagaaatgccccagtgttatttattccccagtgactccagggtgacaaggcctgcttgactttccagaatgaccttgagttaacaggacagttgatggagccccagggtttacatgaagcagaaccttctttggttccatgttgactgacttatggcatgactcttcaaccccgaggtccctgttgtcagatctattgtttgttgcactaaaatgaggatccagggcagcaggccagagaaagcaaaggtgcactccagactctgggggtggacatctgaccccaagggggctgctgctcctctcttgggtagggtagtggctggggtggagtgggaagkgagcattgcagcctaagaagaaggccagagagggaaaaggcaggtgcttttggcagagaccataagagaaacctgccaaggagcatccttggcagtgggaatgttctttctgctctatactgtggcctgcaggagggttggagtgctcttcccactccagctgacagccacaccgtggcagcttgctgggctttgggaagtttgctgtgctttggaacaatcacagggaatggccacaaacctgcccgcctaagaccctgaatccgtacttgggtcacatgactctcattttatttacagctgtgctccacactcagaaaattccctggggtcaccttctagttgcccccattcccagcctgactagaactcctgtcttctttctccatggagcctacctctgtctgagacaggtgcctaacctgggacctgtggtcatgtgagtctgggatattctttagcttacctgggcacagacagaattttccatttattaagcagtacagatgtttttcatccattcctaatcaaattctgtctggggacgaagggttggacgggatgacctccagaagtcccttcaatttctagtacctgtgactcttagccctcaccacagccttctaaattcccaaatcctagactgctcctgggcattagcaaggcagagcctttttacctggcctagaaagggcaaggggtgaggataggacagagggattttgttcaagtttgctgcaacccaagtggacgttaggccaggccttatctgaaaggccagcagctgatgctgtactaacccagtctttcttcactctggcttcaaaaagccacagcagagcattgtcaccgcaggtccccatgctgctcccctaaagccaggctcaggagaagccagtgtctaggcactgagcagggatctgccccctagttcaggtccaaattcaccttcccctaaaccccaagcttcccaacagatcatatggtaggaccctcgagagccttacttcaaagtgcctgggctcagcctggtttctgggtgctagatccagcccaaacctgggaaggccagccttgtacagtctgctcctcttgttcctgaaatgtgtttccttttcaggagatggggaataatttccttcaggcagctgaaattcaccaagaacagcgggtacttatttctcaagctgtgccttccctttctaagcaaccacactgcttggcccttcaagggtcagggtgagacgtgatgggctaggcctccgttgtctggttgctaatgacagccttgcaacccaaggtgaggtgaactccaggcatgtgtctggccctaactcctataaagtgcctcggacagtccgcagttgtagcagaaaccaacaagaaccactccttcatgtttggaaaataatttctcttgtattatctcctttgaagaaggcaaggctgataatatgacaaacatcattgtttagatgaggctcagagaggtagcactctcagagtgttttgaccagtttaagccgcagacctggagcttcagccaggtctgactccaaagctgttccattacaccacagcattgtgtggaatttgaggtctagagagaaccaataaaagtggtaattgggaactgaaatccttgagagttccggggagaaacccagagatgcctgatttcattcctcgatggtaatacccgtcctctcggctgccaggggctctgtggcaaaaagagtcagacatttctttggaaaacagcgaacagccttagagctcttgtgttcagaagaatcttcctggcacaatgttggagcagcaggcctctgggacccacagaacttgtggcctttatgttctttcacccatcctaggaaccagccaaccatcatgtgtagagcccctactgtgggcaaagtcctcctttcattaccctacagacagcttacaggagccagcctgcttcccacaactactagtgtgactccttatctctttccaccataccttagagactttgatactaccagggtctctcagggatggagggaagacctgaaagagaggactggttctgaggccagaaaggtgtgaggagagaggaggaaaagtcttcctaattgtgcccctaaagagcatcctgataccattctattctccagacatggaggggatgataaaggaaataggatctccactggacccttgattcattctgaaccctccaaaggaactctaagagggcgagggatgatgagggaagcaataggtagctggggagccctattgctgctaagtcattggcaaagtgcaaagcaatttactgatgagagaatgtggaaatagatgtgcagtttggaattatgttggtgtnaatttgccagaggaccaatgcttgcatggagaatggacgaggacatttgtgggcaagcagatgacagaggtttgaaggagaatggcatggcaggagtctctgccagttacttgggcttcaacagccaagctggcacaaaagacagctggcggaggctgctcggctactggttacctggagaagtagtatttgcctatttcccccttcatccatcctgagccaaatttcntttgctgaacaggaaagagcyaggaaccctggaggtaaacaaagactttgancctgtntnagtgtatgtgtttntgtaacttcctgtggagtgcaaatagattcagagaaatttagagctaaaaaggcccttagagggaatctagcccaacctacattccaccctgttacttatgtagaaactgaggcccagagagggaagatgacctgccccaagtggtgagcaagcaccaacctccagactcagcagagtgagggggtaaagcagttcctgtcccacatggccatcttctttcttccacccacaaactccaggctggaagtacttggcccccttcaggagcctggccaggcagggagagagtagctgcagccttcatcagaactcttcctcctcccaaggcattctcccagctctagcctctggactggaaagcacaagactggcccagtgccagcaagtccttaggctactgtaatgctgcctcaggacccatccctgcctggaggctcctctaggccctgtgagcacaaagaagaaagctgatttttgtcttttaatccatttcaggactctctccaggagggctcggggtgtgtcatttctatattcctccagctgggattggggggtgggctttgttgtgagaatggcctggagcaggcccaatgctgcttttgggggtcagcatccagtgtgagatactgtgtatataaactatatataatgtatataaactgggatgtaagtttgtgtaaattaatgttttattctttgcaaataaaacgctttccccgtcaaaaaaaaaaaaaaaaaaaaaaaaaaaaaaaaaaaaa

SEQ ID NO: 7 - DNA, 972 bp, Homo sapiens
tctagactcaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagatgttctaga

SEQ ID NO: 8 - DNA, 3362 bp, Homo sapiens
ccatatcttcatcttccctctacccagattgtgaagatggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttgaggggctgatggcaatatctaaaaccaggcaccagcatgaacaccaagctgggggtggacagggcatggattcttcattgcaagtgaaggagcctcccagctcagccacgtgggatgtgacaagaagcagatcctggccctcccgcccccacccctcagggatatttaaaacttattttatataccagttaatcttatttatccttatattttctaaattgcctagccgtcacaccccaagattgccttgagcctactaggcacctttgtgagaaagaaaaaatagatgcctcttcttcaagatgcattgtttctattggtcaggcaattgtcataataaacttatgtcattgaaaacggtacctgactaccatttgctggaaatttgacatgtgtgtggcattatcaaaatgaagaggagcaaggagtgaaggagtggggttatgaatctgccaaaggtggtatgaaccaacccctggaagccaaagcggcctctccaaggttaaattgattgcagtttgcatattgcctaaatttaaactttctcatttggtgggggttcaaaagaagaatcagcttgtgaaaaatcaggacttgaagagagccgtctaagaaataccacgtgctttttttctttaccattttgctttcccagcctccaaacatagttaatagaaatttcccttcaaagaactgtctggggatgtgatgctttgaaaaatctaatcagtgacttaagagagattttcttgtatacagggagagtgagataacttattgtgaagggttagctttactgtacaggatagcagggaactggacatctcagggtaaaagtcagtacggattttaatagcctggggaggaaaacacattctttgccacagacaggcaaagcaacacatgctcatcctcctgcctatgctgagatacgcactcagctccatgtcttgtacacacagaaacattgctggtttcaagaaatgaggtgatcctattatcaaattcaatctgatgtcaaatagcactaagaagttattgtgccttatgaaaaataatgatctctgtctagaaataccatagaccatatatagtctcacattgataattgaaactagaagggtctatatcagcctatgccagggcttcaatggaatagtatccccttatgtttagttgaaatgtccccttaacttgatataatgtgttatgcttatggcgctgtgacaatctgatttttcatgtcaacttccagatgatttgtaacttctctgtgccaaaccttttataaacataaatttttgagatatgtattttaaaattgtagcacatgtttccctgacattttcaatagaggatacaacatcacagaatctttctggatgattctgtgttatcaaggaattgtactgtgctacaattatctctagaatctccagaaaggtggagggctgttcgcccttacactaaatggtctcagttggatttttttttcctgttttctatttcctcttaagtacaccttcaactatattcccatccctctattttaatctgttatgaaggaaggtaaataaaaatgctaaatagaagaaattgtaggtaaggtaagaggaatcaagttctgagtggctgccaaggcactcacagaatcataatcatggctaaatatttatggagggcctactgtggaccaggcactggctaaatacttacatttacaagaatcattctgagacagatattcaatgatatctggcttcactactcagaagattgtgtgtgtgtttgtgtgtgtgtgtgtgtgtgtatttcactttttgttattgaccatgttctgcaaaattgcagttactcagtgagtgatatccgaaaaagtaaacgtttatgactataggtaatatttaagaaaatgcatggttcatttttaagtttggaatttttatctatatttctcacagatgtgcagtgcacatgcaggcctaagtatatgttgtgtgtgtttgtctttgacgtcatggtcccctctcttaggtgctcactcgctttgggtgcacctggcctgctcttcccatgttggcctctgcaaccacacagggatatttctgctatgcaccagcctcactccaccttccttccatcaaaaatatgtgtgtgtgtctcagtccctgtaagtcatgtccttcacagggagaattaacccttcgatatacatggcagagttttgtgggaaaagaattgaatgaaaagtcaggagatcagaattttaaatttgacttagccactaactagccatgtaaccttgggaaagtcatttcccatttctgggtcttgcttttctttctgttaaatgagaggaatgttaaatatctaacagtttagaatcttatgcttacagtgttatctgtgaatgcacatattaaatgtctatgttcttgttgctatgagtcaaggagtgtacacttctcctttactatgttgaatgtatttttttctggacaagcttacatcttcctcagccatctttgtgagtccttcaagagcagttatcaattgttagttagatattttctatttagagaatgcttaagggattccaatcccgatccaaatcataatttgttcttaagtatactgggcaggtcccctattttaagtcataattttgtatttagtgctttcctggctctcagagagtattaatattgatattaataatatagttaatagtaatattgctatttacatggaaacaaataaaagatctcagaattc

SEQ ID NO: 9 - DNA, 534 bp, Homo sapiens
atgtgtttgagccacttggaaaatatgcctttaagccattcaagaactcaaggagctcagagatcatcctggaagctgtggctcttttgctcaatagttatgttgctatttctttgctccttcagttggctaatctttatttttctccaattagagactgctaaggagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctag

SEQ ID NO: 10 - DNA, 1906 bp, Homo sapiens
ccaagtcacatgattcaggattcagggggagaatccttcttggaacagagatgggcccagaactgaatcagatgaagagagataaggtgtgatgtggggaagactatataaagaatggacccagggctgcagcaagcactcaacggaatggcccctcctggagacacagccatgcatgtgccggcgggctccgtggccagccacctggggaccacgagccgcagctatttctatttgaccacagccactctggctctgtgccttgtcttcacggtggccactattatggtgttggtcgttcagaggacggactccattcccaactcacctgacaacgtccccctcaaaggaggaaattgctcagaagacctcttatgtatcctgaaaagagctccattcaagaagtcatgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagactgaacagtttctcttggccttcaggaagaaagcgcctctctaccatacagtatttcatccctccaaacacttgggcaaaaagaaaactttagaccaagacaaactacacagggtattaaatagtatacttctccttctgtctcttggaaagatacagctccagggttaaaaagagagtttttagtgaagtatctttcagatagcaggcagggaagcaatgtagtgtggtgggcagagccccacacagaatcagaagggatgaatggatgtcccagcccaaccactaattcactgtatggtcttgatctatttcttctgttttgagagcctccagttaaaatggggcttcagtaccagagcagctagcaactctgccctaatgggaaatgaaggggagctgggtgtgagtgtttacactgtgcccttcacgggatacttcttttatctgcagatggcctaatgcttagttgtccaagtcgcgatcaaggactctctcacacaggaaacttccctatactggcagatacacttgtgactgaaccatgcccagtttatgcctgtctgactgtcactctggcactaggaggctgatcttgtactccatatgaccccacccctaggaacccccagggaaaaccaggctcggacagccccctgttcctgagatggaaagcacaaatttaatacaccaccacaatggaaaacaagttcaaagacttttacttacagatcctggacagaaagggcataatgagtctgaagggcagtcctccttctccaggttacatgaggcaggaataagaagtcagacagagacagcaagacagttaacaacgtaggtaaagaaatagggtgtggtcactctcaattcactggcaaatgcctgaatggtctgtctgaaggaagcaacagagaagtggggaatccagtctgctaggcaggaaagatgcctctaagttcttgtctctggccagaggtgtggtatagaaccagaaacccatatcaagggtgactaagcccggcttccggtatgagaaattaaacttgtatacaaaatggttgccaaggcaacataaaattataagaattc

SEQ ID NO: 11 - DNA, 2785 bp, Homo sapiens
  misc_feature (49)..(49): n = a, t, c or g
agtgcagtatctcatggaggtgtttggatgtctcttcctgtggggggtnccaaagcccatgtctcttggcattttctttcagattctatcagccctctctctttctctcctgtctctctctttcattcatacactgagtcattcagagatggcttctctccaactcggagctgcaagtaattctggatctggtcacacacacaaagtccccagagttgccaatttatctagttcatctgtgcctgttcaagatgatgtaactaaacatttaccttcagggaggtgtttccaaagaattttcatcgatatatagaaatcaagagaaaatccatactatcaccaaatcaagagaaattccatactatcaccagttggccaactttccaagtctagtgcagaaatccaaggcacctcacacctagagttcctatacctctgagactccagaggaaagaacaagacagtgcagaaggatatgttagaacccactgaaaacctagaaggttaaaaaggaagcataccctcctgacctataagaaaattttcagtctgcagggggatatccttgtggcccaagacattggtgttatcatttgactaagaggaaattatttgtggtgagctctgagtgaggattaggaccagggagatgccaagtttctatcacttacctcatgcctgtaagacaagtgttttgttccaattgatgaatggggataaaacagttcagccaatcacttatggggcaaagaatgggaatttgaagggtctggtgcctggccttgtcatacgtaaacaagagaggcatcgatgagttttatctgagtcatttgggaaaggataattcttgcagcaagccattttcctaaacacagaagaatagggggattccttaaccttcattgttctccaggatcataggtctcaggtaaaattaaaaattttcaggtcagaccactcagtctcagaaaggcaaagtaatttgccccaggtcactagtccaagatgttattctctttgaacaaatgtgtatgtccagtcacatattcttcattcattcctccccaaagcagtttttagctgttaggtatattcgatcactttagtctattttgaaaatgatatgagacgctttttaagcaaagtctacagtttcccaatgagaaaattaatcctctttcttgtctttccagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactataggaggagagcaaatatcattatatgaaagtcctctgccaccgagttcctaattttctttgttcaaatgtaattataaccaggggttttcttggggccgggagtagggggcattccacagggacaacggtttagctatgaaatttggggccaaaatttcacacttcatgtgccttactgatgagagtactaactggaaaaaggctgaagagagcaaatatattattaagatgggttggaggattggcgagtttctaaatattaagacactgatcactaaatgaatggatgatctactcgggtcaggattgaaagagaaatatttcaacacctcctgctatacaatggtcaccagtggtccagttattgttcaatttgatcataaatttgcttcaattcaggagctttgaaggaagtccaaggaaagctctagaaaacagtataaactttcagaggcaaaatccttcaccaatttttccacatactttcatgccttgcctaaaaaaaatgaaaagagagttggtatgtctcatgaatgttcacacagaaggagttggttttcatgtcatctacagcatatgagaaaagctacctttcttttgattatgtacacagatatctaaataaggaagtatgagtttcacatgtatatcaaaaatacaacagttgcttgtattcagtagagttttcttgcccacctattttgtgctgggttctaccttaacccagaagacactatgaaaaacaagacagactccactcaaaatttatatgaacaccactagatacttcctgatcaaacatcagtcaacatactctaaagaataactccaagtcttggccaggcgcagtggctcacacctgtaatcccaacactttgggaggccaaggtgggtggatcatctaaggccgggagttcaagaccagcctgaccaacgtggagaaaccccatctctactaaaaatacaaaattagccgggcgtggtagcgcatggctgtaatcctggctactcaggaggccgaggcagaagaattgcttgaactggggaggcagaggttgcggtgagcccagatcgcgccattgcactccagcctgggtaacaagagcaaaactctgtccaaaaaaaaaaaaaaaaaaaa

SEQ ID NO: 12 - DNA, 1169 bp, Homo sapiens
gaggttgaaggacccaggcgtgtcagccctgctccagagaccttgggcatggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggctgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggaggaggtggtcgtccgtgtgctggatgaacgcctggttcgactgcgtgatggtacccggtcttacttcggggctttcatggtgtgaaggaaggagcgtggtgcattggacatgggtctgacacgtggagaactcagagggtgcctcaggggaaagaaaactcacgaagcagaggctgggcgtggtggctctcgcctgtaatcccagcactttgggaggccaaggcaggcggatcacctgaggtcaggagttcgagaccagcctggctaacatggcaaaaccccatctctactaaaaatacaaaaattagccggacgtggtggtgcctgcctgtaatccagctactcaggaggctgaggcaggataattttgcttaaacccgggaggcggaggttgcagtgagccgagatcacaccactgcactccaacctgggaaacgcagtgagactgtgcctcaaaaaaaaaaaaaaaaaaaaaaaaaaaaa

SEQ ID NO: 13 - DNA, 1619 bp, Homo sapiens
gtcatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataacgcccagcctgggtgcagcccacctggacagagtccgaatcctactccatccttcatggagacccctggtgctgggtccctgctgctttctctacctcaaggggcttggcaggggtccctgctgctgacctccccttgaggaccctcctcacccactccttccccaagttggaccttgatatttattctgagcctgagctcagataatatattatatatattatatatatatatatatttctatttaaagaggatcctgagtttgtgaatggacttttttagaggagttgttttggggggggggtcttcgacattgccgaggctggtcttgaactcctggacttagacgatcctcctgcctcagcctcccaagcaactgggattcatcctttctattaattcattgtacttatttgcctatttgtgtgtattgagcatctgtaatgtgccagcattgtgcccaggctagggggctatagaaacatctagaaatagactgaaagaaaatctgagttatggtaatacgtgaggaatttaaagactcatccccagcctccacctcctgtgtgatacttgggggctagcttttttctttctttcttttttttgagatggtcttgttctgtcaaccaggctagaatgcagcggtgcaatcatgagtcaatgcagcctccagcctcgacctcccgaggctcaggtgatcctcccatctcagcctctcgagtagctgggaccacagttgtgtgccaccacacttggctaactttttaatttttttgcggagacggtattgctatgttgccaaggttgtttacatgccagtacaatttataataaacactcatttttcc

SEQ ID NO: 14 - DNA, 926 bp, Homo sapiens
ccagagaggggcaggcttgtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgtctcccagcgcctgacgcccctggcccgaggggacacactctgcaccaacctcactgggacacttttgccttcccgaaacactgatgagaccttctttggagtgcagtgggtgcgcccctgaccactgctgctgattagggttttttaaattttattttattttatttaagttcaagagaaaaagtgtacacacaggggccacccggggttggggtgggagtgtggtggggggtagtttgtggcaggacaagagaaggcattgagctttttctttcattttcctattaaaaaatacaaaaatcaaaacaaaaaaaa

SEQ ID NO: 15 - DNA, 894 bp, Homo sapiens
cagtctcaatgggggcactggggctggagggcaggggtgggaggctccaggggaggggttccctcctgctagctgtggcaggagccacttctctggtgaccttgttgctggcggtgcctatcactgtcctggctgtgctggccttagtgccccaggatcagggaggactggtaacggagacggccgaccccggggcacaggcccagcaaggactggggtttcagaagctgccagaggaggagccagaaacagatctcagccccgggctcccagctgcccacctcataggcgctccgctgaaggggcaggggctaggctgggagacgacgaaggaacaggcgtttctgacgagcgggacgcagttctcggacgccgaggggctggcgctcccgcaggacggcctctattacctctactgtctcgtcggctaccggggccgggcgccccctggcggcggggacccccagggccgctcggtcacgctgcgcagctctctgtaccgggcggggggcgcctacgggccgggcactcccgagctgctgctcgagggcgccgagacggtgactccagtgctggacccggccaggagacaagggtacgggcctctctggtacacgagcgtggggttcggcggcctggtgcagctccggaggggcgagagggtgtacgtcaacatcagtcaccccgatatggtggacttcgcgagagggaagaccttctttggggccgtgatggtggggtgagggaatatgagtgcgtggtgcgagtgcgtgaatattgggggcccggacgcccaggaccccatggcagtgggaaaaatgtaggagactgtttggaaattgattttgaacctgatgaaaataaagaatggaaagcttcagtgctgccgataaa

SEQ ID NO: 16 - DNA, 1306 bp, Homo sapiens
cacagccccccgcccccatggccgcccgtcggagccagaggcggagggggcgccggggggagccgggcaccgccctgctggtcccgctcgcgctgggcctgggcctggcgctggcctgcctcggcctcctgctggccgtggtcagtttggggagccgggcatcgctgtccgcccaggagcctgcccaggaggagctggtggcagaggaggaccaggacccgtcggaactgaatccccagacagaagaaagccaggatcctgcgcctttcctgaaccgactagttcggcctcgcagaagtgcacctaaaggccggaaaacacgggctcgaagagcgatcgcagcccattatgaagttcatccacgacctggacaggacggagcgcaggcaggtgtggacgggacagtgagtggctgggaggaagccagaatcaacagctccagccctctgcgctacaaccgccagatcggggagtttatagtcacccgggctgggctctactacctgtactgtcaggtgcactttgatgaggggaaggctgtctacctgaagctggacttgctggtggatggtgtgctggccctgcgctgcctggaggaattctcagccactgcggccagttccctcgggccccagctccgcctctgccaggtgtctgggctgttggccctgcggccagggtcctccctgcggatccgcaccctcccctgggcccatctcaaggctgcccccttcctcacctacttcggactcttccaggttcactgaggggccctggtctccccacagtcgtcccaggctgccggctcccctcgacagctctctgggcacccggtcccctctgccccaccctcagccgctctttgctccagacctgcccctccctctagaggctgcctgggcctgttcacgtgttttccatcccacataaatacagtattcccactcttatcttacaactcccccaccgcccactctccacctcactagctccccaatccctgaccctttgaggcccccagtgatctcgactcccccctggccacagacccccagggcattgtgttcactgtactctgtgggcaaggatgggtccagaagaccccacttcaggcactaagaggggctggacctggcggcaggaagccaaagagactgggcctaggccaggagttcccaaatgtgaggggcgagaaacaagacaagctcctcccttgagaattccctgtggatttttaaaacagatattatttttattattattgtgacaaaatgttgataaatggatattaaatagaataagtcag

SEQ ID NO: 17 - DNA, 1348 bp, Homo sapiens
ggtacgaggcttcctagagggactggaacctaattctcctgaggctgagggagggtggagggtctcaaggcaacgctggccccacgacggagtgccaggagcactaacagtacccttagcttgctttcctcctccctcctttttattttcaagttcctttttatttctccttgcgtaacaaccttcttcccttctgcaccactgcccgtacccttacccgccccgccacctccttgctaccccactcttgaaaccacagctgttggcagggtccccagctcatgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgattgtgttataaaaagtggctcccagcttggaagaccagggtgggtacatactggagacagccaagagctgagtatataaaggagagggaatgtgcaggaacagaggcatcttcctgggtttggctccccgttcctcacttttcccttttcattcccaccccctagactttgattttacggatatcttgcttctgttccccatggagctccgaattcttgcgtgtgtgtagatgaggggcgggggacgggcgccaggcattgttcagacctggtcggggcccactggaagcatccagaacagcaccaccatctta

SEQ ID NO: 18 - DNA, 1090 bp, Homo sapiens
taactctcctgaggggtgagccaagccctgccatgtagtgcacgcaggacatcaacaaacacagataacaggaaatgatccattccctgtggtcacttattctaaaggccccaaccttcaaagttcaagtagtgatatggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcctggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtgacctacttacaccatgtctgtagctattttcctccctttctctgtacctctaagaagaaagaatctaactgaaaataccaaaaaaaaaaaaaaaaaa

SEQ ID NO: 19 - protein, 316 aa, Homo sapiens
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENAGLQDSTLESEDTLPDSCRRMKQAFQGAVQKELQHIVGPQRFSGAPAMMEGSWLDVAQRGKPEAQPFAHLTINAASIPSGSHKVTLSSWYHDRGWAKISNMTLSNGKLRVNQDGFYYLYANICFRHHETSGSVPTDYLQLMVYVVKTSIKIPSSHNLMKGGSTKNWSGNSEFHFYSINVGGFFKLRAGEEISIQVSNPSLLDPDQDATYFGAFKVQDID

SEQ ID NO: 20 - protein, 24 aa, Homo sapiens
NAASIPSGSHKVTLSSWYHDRGWA

SEQ ID NO: 21 - protein, 10 aa, Homo sapiens
HETSGSVPTD

SEQ ID NO: 22 - protein, 7 aa, Homo sapiens
SIKIPSS

SEQ ID NO: 23 - protein, 9 aa, Homo sapiens
KNWSGNSEF

SEQ ID NO: 24 - protein, 34 aa, Homo sapiens
TRGRSNTLSSPNSKNEKALGRKINSWESSRSGHS

SEQ ID NO: 25 - protein, 10 aa, Homo sapiens
QEEIKENTKN

SEQ ID NO: 26 - protein, 6 aa, Homo sapiens
TSYPDP

SEQ ID NO: 27 - protein, 9 aa, Homo sapiens
SCWSKDAEY

SEQ ID NO: 28 - protein, 18 aa, Homo sapiens
EASSKTTSVLQWAEKGYY

SEQ ID NO: 29 - protein, 6 aa, Homo sapiens
NREASS

SEQ ID NO: 30 - protein, 6 aa, Homo sapiens
SPGRFE

SEQ ID NO: 31 - protein, 7 aa, Homo sapiens
HSSAKPC

SEQ ID NO: 32 - protein, 17 aa, Homo sapiens
NHQVEEQLEWLSQRANA

SEQ ID NO: 33 - protein, 5 aa, Homo sapiens
QGCPD

SEQ ID NO: 34 - protein, 7 aa, Homo sapiens
AISYQEK

SEQ ID NO: 35 - protein, 14 aa, Homo sapiens
PCPKDTPEGAELKP

SEQ ID NO: 36 - protein, 17 aa, Homo sapiens
DPSKQNSLLWRANTDRA

SEQ ID NO: 37 - protein, 10 aa, Homo sapiens
KAYSPKATSS

SEQ ID NO: 38 - protein, 7 aa, Homo sapiens
SSQYPFH

SEQ ID NO: 39 - protein, 8 aa, Homo sapiens
VYPGLQEP

SEQ ID NO: 40 - protein, 17 aa, Homo sapiens
ETRVTVPNVPIRFTKIF

SEQ ID NO: 41 - protein, 1 aa, Homo sapiens
D

SEQ ID NO: 42 - protein, 4 aa, Homo sapiens
YQEK

SEQ ID NO: 43 - protein, 24 aa, Homo sapiens
NATDIPSGSHKVSLSSWYHDRGWA

SEQ ID NO: 44 - protein, 10 aa, Homo sapiens
HETSGDLATE

SEQ ID NO: 45 - protein, 7 aa, Homo sapiens
SIKIPSS

SEQ ID NO: 46 - protein, 9 aa, Homo sapiens
KYWSGNSEF

SEQ ID NO: 47 - protein, 17 aa, Homo sapiens
NPQAEGQLQWLNRRANA

SEQ ID NO: 48 - protein, 5 aa, Homo sapiens
QGCPS

SEQ ID NO: 49 - protein, 7 aa, Homo sapiens
AVSYQTK

SEQ ID NO: 50 - protein, 14 aa, Homo sapiens
PCQRETPEGAEAKP

SEQ ID NO: 51 - DNA, 2201 bp, Homo sapiens
ggccaaagccgggctccaagtcggcgccccacgtcgaggctccgccgcagcctccggagttggccgcagacaagaaggggagggagcgggagagggaggagagctccgaagcgagagggccgagcgccatgcgccgcgccagcagagactacaccaagtacctgcgtggctcggaggagatgggcggcggccccggagccccgcacgagggccccctgcacgccccgccgccgcctgcgccgcaccagccccccgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggccccaacggtacacgactcagtatccatgctcttgaccttgtagagaacacgcgtatttacctgccagtgggagatgttagactcatggtgtgttacacaatggtttttaaattttgtaatgaattcctagaattaaaccagattggagcaattacgggttgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatttgtcaagagtagttatataattgttgaacaggtgtttttccacaagtgccgcaaattgtacctttttttttttttcaaaatagaaaagttattagtggtttatcagcaaaaaagtccaattttaatttagtaaatgttattttatactgtacaataaaaacattgcctttgaatgttaattttttggtacaaaaataaatttatatgaaaaaaaaaaaaaaaaaaaaaaaaaaaaaaaa

(19) United States
(12) Patent Application Publication
(10) Pub. No.: US 2003/0109444 A1
(43) Pub. Date: Jun. 12, 2003

(54) Bone anti-resorptive compounds

(76) Inventors: Jonathan Lam, West Memphis, AR (US); F. Patrick Ross, Overland, MO (US); Steven L. Teitelbaum, St. Louis, MO (US)

Correspondence Address: SONNENSCHEIN NATH & ROSENTHAL, P.O. BOX 061080, WACKER DRIVE STATION, CHICAGO, IL, 60606-1080, US

(21) Appl. No.: 10/272,328

(22) Filed: Oct. 15, 2002

Related U.S. Application Data
(60) Provisional application No. 60/329,360, filed on Oct. 15, 2001.
(60) Provisional application No. 60/329,231, filed on Oct. 12, 2001.
(60) Provisional application No. 60/329,393, filed on Oct. 15, 2001.
(60) Provisional application No. 60/328,876, filed on Oct. 12, 2001.

(51) Int. Cl. A61K 38/17; C07K 14/47; C12P 21/02; C12N 5/06

(52) U.S. Cl. 514/12; 530/350; 435/69.1; 435/320.1; 435/325; 536/23.5

(57) ABSTRACT

The present invention relates to polypeptides that bind to RANK and comprise amino acid sequences of RANKL external surface loops. The invention also relates to fragments, analogs, and derivatives of such polypeptides.

The present invention further relates to compositions comprising the polypeptides described herein. Also included are methods for inhibiting osteoclast differentiation, methods for inhibiting bone resorption, and methods for competitively inhibiting RANKL activity. The invention also provides methods for treating diseases or conditions which are at least partially characterized by loss of bone mass.

This application is related to and claims the benefit of the following U.S. applications, which are incorporated herein by reference: Ser. No. 60/277,855 filed Mar. 22, 2001; Ser. No. 10/105,057 filed Mar. 22, 2002; Ser. No. 60/311,163 filed Aug. 9, 2001; Ser. No. 10/215,446 filed Aug. 9, 2002; Ser. No. 60/329,231 filed Oct. 12, 2001; Ser. No. 60/329,393 filed Oct. 15, 2001; Ser. No. 60/329,360 filed Oct. 15, 2001; Ser. No. 60/328,876 filed Oct. 12, 2001; U.S. non-provisional entitled RANKL Mimics and Uses Thereof, Lam, et al. filed Oct. 15, 2002; and U.S. non-provisional entitled Methods for Screening Osteogenic Compounds, Lam, et al. filed Oct. 15, 2002.

This invention was made in part with Government support under National Institutes of Health Grants AR32788, AR46123 and DE05413. The Government has certain rights in the invention.

### FIELD OF THE INVENTION

The present invention relates to polypeptides that bind to RANK and whose amino acid sequences comprise one or more external surface loops AA″, CD, EF, and DE of RANKL. Further provided are fragments, analogs, and derivatives of said polypeptides.

The invention also relates to pharmaceutical compositions comprising the polypeptides provided herein, and/or fragments, analogs, and derivatives thereof. The invention further provides methods for inhibiting osteoclast differentiation and methods for competitively inhibiting RANKL comprising administering an effective amount of said compositions. Also provided are methods for inhibiting bone resorption and methods for treating diseases or conditions which are at least partially characterized by loss of bone mass.

### BACKGROUND

Various conditions and diseases which manifest themselves in bone loss or thinning are a critical and growing health concern. It has been estimated that as many as 30 million Americans and 100 million worldwide are at risk for osteoporosis alone. Mundy et al., Science, 286: 1946-1949 (1999). Other conditions known to involve bone loss include juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, corticosteroid treatment, metastatic bone diseases, periodontal bone loss, bone loss due to cancer, age-related loss of bone mass, and other forms of osteopenia. Additionally, new bone formation is needed in many situations, e.g., to facilitate bone repair or replacement for bone fractures, bone defects, plastic surgery, dental and other implantations and in other such contexts.

Bone is a dense, specialized form of connective tissue. Bone matrix is formed by osteoblast cells located at or near the surface of existing bone matrix. Bone is resorbed (eroded) by another cell type known as the osteoclast (a type of macrophage). These cells secrete acids, which dissolve bone minerals, and hydrolases, which digest its organic components. Thus, bone formation and remodeling is a dynamic process involving an ongoing interplay between the creation and erosion activities of osteoblasts and osteoclasts. Alberts, et al., Molecular Biology of the Cell, Garland Publishing, New York (3rd ed. 1994), pp. 1182-1186.

Present forms of clinically-approved bone loss therapy are primarily anti-resorptive, in that they inhibit bone resorption processes. Among the agents which have been used or suggested for treatment of osteoporosis because of their claimed ability to inhibit bone resorption are estrogen, selective estrogen receptor modulators (SERMs), calcium, calcitriol, calcitonin (Sambrook, P. et al., N.Engl.J.Med. 328:1747-1753), alendronate (Saag, K. et al., N.Engl.J.Med. 339:292-299) and other bisphosphonates. Luckman et al., J. Bone Min. Res. 13, 581 (1998). However, currently-available anti-resorptives may have undesired effects relating to their impact on the inhibition of bone resorption/remodeling or other unwanted side effects.

As a result, it would be very desirable to obtain other compounds for treatments of bone loss diseases. A key development in the field of bone cell biology is the recent discovery that RANK ligand (RANKL, also known as osteoprotegerin ligand (OPGL), TNF-related activation induced cytokine (TRANCE), and osteoclast differentiation factor (ODF)), expressed on stromal cells, osteoblasts, activated T-lymphocytes and mammary epithelium, is the unique molecule essential for differentiation of macrophages into osteoclasts. Lacey, et al., Cell 93: 165-176 (1998) (Osteoprotegerin Ligand Is a Cytokine that Regulates Osteoclast Differentiation and Activation.) The cell surface receptor for RANKL is RANK, Receptor Activator of Necrosis Factor (NF)-kappa B. RANKL is a type-2 transmembrane protein with an intracellular domain of less than about 50 amino acids, a transmembrane domain of about 21 amino acids, and an extracellular domain of about 240 to 250 amino acids. RANKL exists naturally in transmembrane and soluble forms. The deduced amino acid sequence for at least the murine, rat and human forms of RANKL and variants thereof are known. See e.g., Anderson, et al., U.S. Pat. No. 6,017,729, Boyle, U.S. Pat. No. 5,843,678, and Xu J. et al., J. Bone Min. Res. (2000/15:2178) which are incorporated herein by reference. Furthermore, we have solved the crystal structure of RANKL ectodomain, as disclosed in application Ser. No. 60/311,163, filed Aug. 9, 2001 and Ser. No. 10/215,446 filed Aug. 9, 2002.

RANKL (OPGL) has been identified as a potent inducer of bone resorption and as a positive regulator of osteoclast development. Lacey et al., supra. In addition to its role as a factor in osteoclast differentiation and activation, RANKL has been reported to induce human dendritic cell (DC) cluster formation. Anderson et al., supra and mammary epithelium development J. Fata et al., “The osteoclast differentiation factor osteoprotegerin ligand is essential for mammary gland development,” Cell, 103:41-50 (2000). Recently, we have determined that RANKL plays a role in anabolic bone formation processes and can be utilized in methods for stimulation of osteoblast proliferation or bone nodule mineralization, as disclosed in applications Ser. No. 60/277,855, filed Mar. 22, 2001 and Ser. No. 10,105,057 filed Mar. 22, 2002. We have also recently determined the precise three-dimensional structure of RANKL's ectodomain, and located the amino acid sequences of RANKL's unique external surface loops which interact with RANK, as disclosed in applications Ser No. 60/311, 163, filed Aug. 9, 2001 and Ser. No. 10/215,446 filed Aug. 9, 2002.

Accordingly, due to the limited success in treatment of bone loss disorders with current medications, a need exists to develop novel therapeutics for treatment of such conditions.

### SUMMARY OF THE INVENTION

Accordingly, among the objects of the invention is the provision of polypeptides comprising one or more of the external surface loops AA″, CD, DE, or EF of RANKL and having the ability to bind to RANK. The RANKL loops correspond to the portions of RANKL molecule (SEQ ID NO 6) described below:

AA″ contains amino acid residues 170-193 (SEQ ID NO 2),

CD contains amino acid residues 224-233 (SEQ ID NO 3),

DE contains amino acid residues 245-251 (SEQ ID NO 4), and

EF contains amino acid residues 261-269 (SEQ ID NO 5).

A polypeptide containing a portion of AA″ loop sequence is also included in the invention, and it includes amino acid residues 175-185 (SEQ ID NO 1). Polypeptides of SEQ ID NO: 7 and SEQ ID NO: 11 are natural occurring variants of the AA″ loop of human RANKL. Both are natural variants of human RANKL. SEQ ID NO: 8, SEQ ID NO: 9 and SEQ ID NO: 10 are surface loop polypeptide sequences of human RANKL loops CD, DE and EF respectively.

In another aspect, the invention encompasses polypeptides that bind to RANK and consist of sequences selected from the group consisting of SEQ ID NO 1-SEQ ID NO 5 and SEQ ID NO: 7-SEQ ID NO: 11.

It is another object of the invention to provide polypeptides comprising external surface loops of RANKL and having the ability to competitively inhibit RANKL.

It is a further object of the invention to provide fragments, analogs, and derivatives of such polypeptides. The methods for obtaining such fragments, analogs, and derivatives are described herein.

In another aspect, the present invention provides pharmaceutical compositions comprising the polypeptides. The compositions may further include pharmaceutically acceptable carriers, adjuvants, solubilizers, stabilizers, and/or anti-oxidants.

The invention also encompasses methods for inhibiting osteoclast differentiation, methods for competitively inhibiting RANKL, and methods for inhibiting bone resorption. Such methods include administration of compositions of the invention. Further provided are methods for treating diseases and conditions which are at least in part characterized by loss of bone mass. In a preferred embodiment, such methods are used to treat osteoporosis, osteolytic bone disease, rheumatoid arthritis, and skeletal metastasis.

Other objects and features will be in part apparent and in part pointed out hereinafter.

### BRIEF DESCRIPTION OF FIGURES

FIG. 1 is a graph depicting the effect of increasing concentration of PSGSHKVTLSS peptide (SEQ ID NO 1) on osteoclast generation as measured by TRAP activity.

Abbreviations and Definitions

To facilitate understanding of the invention, a number of terms are defined below:

The amino acid notations used herein for the twenty genetically encoded L-amino acids are conventional and are abbreviated as follows:

| | One | |
|---|---|---|
| | Letter | Three-Letter |
| Amino Acid | Symbol | Symbol |
| Alanine | A | Ala |
| Arginine | R | Arg |
| Asparagine | N | Asn |
| Aspartic | D | Asp |
| acid | | |
| Cysteine | C | Cys |
| Glutamine | Q | Gln |
| Glutamicacid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

As used herein, unless specifically delineated otherwise, the three-letter and one-letter amino acid abbreviations designate amino acids in either the D-configuration or the L-configuration. For example, Arg designates D-arginine and L-arginine, and R designates D-arginine and L-arginine.

Unless noted otherwise, when polypeptide sequences are presented as a series of one-letter and/or three-letter abbreviations, the sequences are presented in the N→C direction, in accordance with common practice. As used herein, “C” refers to the alpha carbon of an amino acid residue. For purposes of determining conservative amino acid substitutions in the various polypeptides described herein and for describing the various peptide and peptide analog compounds, the amino acids can be conveniently classified into two main categories—hydrophilic and hydrophobic—depending primarily on the physical-chemical characteristics of the amino acid side chain. These two main categories can be further classified into subcategories that more distinctly define the characteristics of the amino acid side chains. For example, the class of hydrophilic amino acids can be further subdivided into acidic, basic and polar amino acids. The class of hydrophobic amino acids can be further subdivided into non-polar and aromatic amino acids. The definitions of the various categories of amino acids are as follows:

“Hydrophilic amino acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Thr (T), Ser (S), His (H), Glu (E), Asn (N), Gln (Q), Asp (D), Lys (K) and Arg (R).

“Acidic amino acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

“Basic amino acid” refers to a hydrophilic amino acid having a side chain pK value of greater than 7. Basic amino acids typically have positively charged side chains at physiological pH due to association with hydronium ion. Genetically encoded basic amino acids include His (H), Arg (R) and Lys (K).

“Polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

“Hydrophobic amino acid” refers to an amino acid exhibiting a hydrophobicity of greater than zero according to the normalized consensus hydrophobicity scale of Eisenberg, 1984, J. Mol. Biol. 179:125-142. Genetically encoded hydrophobic amino acids include Pro (P), Lle (I), Phe (F), Val (V), Leu (L), Trp (W), Met (M), Ala (A), Gly (G) and Tyr (Y).

“Aromatic amino acid” refers to a hydrophobic amino acid with a side chain having at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, substituted (C₁-C₆) alkyl, (C₂-C₆) alkenyl, substituted (C₂-C₆) alkenyl, (C₂-C₆) alkynyl, substituted (C₂ C₆) alkynyl, (C₅-C₂₀) aryl, substituted (C₅-C₂₀) aryl, (C₆-C₂₆) arylalkyl, substituted (C₆-C₂₆) arylalkyl, 5-20 membered heteroaryl, substituted 5-20 membered heteroaryl, 6-26 membered heteroarylalkyl or substituted 6-26 membered heteroarylalkyl. Genetically encoded aromatic amino acids include His (H), Phe (F), Tyr (Y) and Trp (W).

“Apolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded apolar amino acids include Leu (L), Val (V), Ile (I), Met (M), Gly (G) and Ala (A).

“Aliphatic amino acid” refers to a hydrophobic amino acid having an aliphatic hydrocarbon side chain. Genetically encoded aliphatic amino acids include Ala (A), Val (V), Leu (L) and Ile (I).

“Hydroxyl-substituted aliphatic amino acid” refers to a hydrophilic polar amino acid having a hydroxyl-substituted side chain. Genetically-encoded hydroxyl-substituted aliphatic amino acids include Ser (S) and Thr (T).

The amino acid residue Cys (C) is unusual in that it can form disulfide bridges with other Cys (C) residues or other sulfanyl-containing amino acids. The ability of Cys (C) residues (and other amino acids with —SH containing side chains) to exist in a peptide in either the reduced free —SH or oxidized disulfide-bridged form affects whether Cys (C) residues contribute net hydrophobic or hydrophilic character to a peptide. While Cys (C) exhibits a hydrophobicity of 0.29 according to the normalized consensus scale of Eisenberg (Eisenberg, 1984, supra), it is to be understood that for purposes of the present invention Cys (C) is categorized as a polar hydrophilic amino acid, notwithstanding the general classifications defined above.

As will be appreciated by those of skill in the art, the above-defined categories are not mutually exclusive. Thus, amino acids having side chains exhibiting two or more physical-chemical properties can be included in multiple categories. For example, amino acid side chains having aromatic moieties that are further substituted with polar substituents, such as Tyr (Y), may exhibit both aromatic hydrophobic properties and polar or hydrophilic properties, and can therefore be included in both the aromatic and polar categories. As another example, His (H) has a side chain that falls within the aromatic and basic categories. The appropriate categorization of any amino acid will be apparent to those of skill in the art, especially in light of the detailed disclosure provided herein.

While the above-defined categories have been exemplified in terms of the genetically encoded amino acids, the amino acid substitutions need not be, and in certain embodiments preferably are not, restricted to the genetically encoded amino acids. Indeed, since many of the compounds described herein may be produced synthetically, they may comprise one or more genetically non-encoded amino acids. Thus, in addition to the naturally occurring genetically encoded amino acids, amino acid residues in the core peptides of structure (1) may be substituted with naturally occurring non-encoded amino acids and synthetic amino acids.

Certain commonly encountered amino acids of which the compounds of the invention may be comprised include, but are not limited to, β-alanine (β-Ala) and other omega-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); naphthylalanine (Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic);β-2-thienylalanine (Thi); methionine sulfoxide (MSO); homoarginine (hArg); N-acetyl lysine (AcLys); 2,4-diaminobutyric acid (Dbu); 2,3-diaminobutyric acid (Dab); p-aminophenylalanine (Phe(pNH₂)); N-methyl valine (MeVal); homocysteine (hCys), homophenylalanine (hPhe) and homoserine (hSer); hydroxyproline (Hyp), homoproline (hPro), N-methylated amino acids and peptoids (N-substituted glycines).

The classifications of the genetically encoded and common non-encoded amino acids according to the categories defined above are summarized in Table 3, below. It is to be understood that Table 3 is for illustrative purposes only and does not purport to be an exhaustive list of amino acid residues that can be used in the invention. Additional amino acids may be found in Fasman, 1989, Practical Handbook of Biochemistry and Molecular Biology, CRC Press, Inc., pp. 3-70, and the references cited therein.

As used herein, a “recombinant nucleic acid” is defined either by its method of production or its structure. In reference to its method of production, e.g., a product made by a process, the process is use of recombinant nucleic acid techniques, e.g., involving human intervention in the nucleotide sequence, typically selection or production. Alternatively, it can be a nucleic acid made by generating a sequence comprising fusion of two fragments which are not naturally contiguous to each other, but is meant to exclude products of nature, e.g., naturally occurring mutants. Thus, for example, products made by transforming cells with any unnaturally occurring vector is encompassed, as are nucleic acids comprising sequences derived using any synthetic oligonucleotide process. Such is often done to replace a codon with a redundant codon encoding the same or a conservative amino acid, while typically introducing or removing a sequence recognition site. Alternatively, it is performed to join together nucleic acid segments of desired functions to generate a single genetic entity comprising a desired combination of functions not found in the commonly available natural forms. Restriction enzyme recognition sites are often the target of such artificial manipulations, but other site specific targets, e.g., promoters, DNA replication sites, regulation sequences, control sequences, or other useful features may be incorporated by design.

As used herein, “polynucleotide” and “oligonucleotide” are used interchangeably and mean a polymer of at least 2 nucleotides joined together by phosphodiester bonds and may consist of either ribonucleotides or deoxyribonucleotides.

As used herein, “sequence” means the linear order in which monomers occur in a polymer, for example, the order of amino acids in a polypeptide or the order of nucleotides in a polynucleotide.

As used herein, “peptide”, “polypeptide” and “protein” are used interchangeably and mean a compound that consists of two or more amino acids that are linked by means of peptide bonds.

As used herein “recombinant protein” means that the protein, whether comprising a native or mutant primary amino acid sequence, is obtained by expression of a gene carried by a recombinant DNA molecule in a cell other than the cell in which that gene and/or protein is naturally found. In other words, the gene is heterologous to the host in which it is expressed. It should be noted that any alteration of a gene, including the addition of a polynucleotide encoding an affinity purification moiety to the gene, makes that gene unnatural for the purposes of this definition, and thus that gene cannot be ‘naturally’ found in any cell.

As used herein, “mutein” includes fragments, derivatives, and analogs of polypeptides.

As used herein, “RANK” refers to RANK protein, recombinant RANK proteins, RANK fusion proteins, analogs, derivatives and mimics thereof.

As used herein, the term “animal” includes human beings.

The phrase “preventing or inhibiting” is being affected either by being inhibited, expressed in another manner, or reduced to such an extent that the observed property is measurably lower than is the case when the treatment is not employed. Measurement of the degree of inhibition can be determined in vitro by methods known to the person skilled in the art.

By the term “an effective amount” is meant an amount of the substance in question which produces a statistically significant effect. For example, an “effective amount” for therapeutic uses is the amount of the composition comprising an active compound herein required to provide a clinically significant increase in healing rates in fracture repair; reversal or inhibition of bone loss in osteoporosis; prevention or delay of onset of osteoporosis; repair or prevention of dental defects; or treatment or inhibition of other bone loss conditions, diseases or defects, including but not limited to those discussed herein above. Such effective amounts will be determined using routine optimization techniques and are dependent on the particular condition to be treated, the condition of the patient, the route of administration, the formulation, and the judgment of the practitioner and other factors evident to those skilled in the art. The dosage required for the compounds of the invention (for example, in osteoporosis) is manifested as that which induces a statistically significant difference in bone mass between treatment and control groups. This difference in bone mass may be seen, for example, as at least 1-2%, or any clinically significant enhancement in bone mass for the treatment group. Other measurements of clinically significant increases in healing may include, for example, tests for breaking strength and tension, breaking strength and torsion, 4-point bending, and other biomechanical tests well known to those skilled in the art. General guidance for treatment regimens is obtained from the experiments carried out in animal models of the disease of interest.

As used herein, “treatment” includes both prophylaxis and therapy. Thus, in treating a subject, the compounds of the invention may be administered to a subject already suffering from loss of bone mass or to prevent or inhibit the occurrence of such condition.

### DETAILED DESCRIPTION

In accordance with the present invention, applicants have discovered that a polypeptide whose sequence represents a subset of the AA″ loop of RANKL acts as a competitive antagonist of RANKL by preventing RANKL from inducing cellular differentiation of osteoclast precursors. Furthermore, the observed inhibition of osteoclast differentiation was dose-dependent.

The U.S. applications Ser. No. 60/311,163 and No. 10/215,446, filed Aug. 9, 2001 and 2002, respectively, disclose the identity of RANKL surfaces that are responsible for binding to RANK, and these include external surface loops AA″, CD, DE, and EF. The external (solvent-accessible) surface loops of RANKL are unique within the TNF family, displaying markedly divergent lengths and conformations: the AA″ loop (residues 170-193 of RANKL protein) bridges strands A and A′, the CD loop (residues 224-233) connects strands C and D, the EF loop (residues 261-269) links strands E and F, and the loop DE (residues 245-251) connects strands D and E. RANKL possesses a longer AA″ loop and a shorter EF loop than the typical TNF family member. The AA″ loop, together with the displacement of the CD loop confers a unique surface to the upper third of the RANKL molecule, whereas a subtle shift of the DE loop shapes the receptor binding groove at the base of RANKL molecule. For a detailed description of RANKL loops and binding specificity of RANK/RANKL interaction see U.S. applications Ser. No. 60/311,163 and No. 10/215,446, filed Aug. 9, 2001 and 2002, respectively.

Thus, applicants have contemplated the use of polypeptides containing RANKL external surface loop sequences and muteins thereof for inhibiting osteoclast differentiation. Consequently, such polypeptides may be used to treat diseases or conditions manifested at least in part by loss of bone mass.

Accordingly, the present invention provides polypeptides comprising one or more of external surface loops AA″, CD, DE, or EF of RANKL and having the ability to bind to RANK. The RANKL loops correspond to the portions of RANKL molecule (SEQ ID NO 6) described below:

AA″ contains amino acid residues 170-193 (SEQ ID NO 2),

CD contains amino acid residues 224-233 (SEQ ID NO 3),

DE contains amino acid residues 245-251 (SEQ ID NO 4), and

EF contains amino acid residues 261-269 (SEQ ID NO 5).

The invention is illustrated by a polypeptide containing a portion of the AA″ loop sequence which includes amino acid residues 175-185 (SEQ ID NO 1).

In one aspect, the RANKL loops correspond to the portions of human RANKL including polypeptides of SEQ ID NO: 7 and SEQ ID NO: 11 which are natural occurring variants of the AA″ loop of human RANKL. Both are natural variants of human RANKL. SEQ ID NO: 8, SEQ ID NO: 9 and SEQ ID NO: 10 are surface loop polypeptide sequences of human RANKL loops CD, DE and EF respectively.

In another aspect, the invention encompasses polypeptides that bind to RANK and consist of sequences selected from the group consisting of SEQ ID NO 1-SEQ ID NO 5 and SEQ ID NO: 7-SEQ ID NO: 11. In another aspect, the invention encompasses concatemers of one or more polypeptides selected from SEQ ID NO 1-SEQ ID NO 5 and SEQ ID NO: 7-SEQ ID NO: 11. In another aspect, the invention encompasses therapeutic combinations of said polypeptides. The invention also provides polypeptides comprising RANKL external surface loops and having the ability to competitively inhibit RANKL.

It will be appreciated that by virtue of the present invention, the above-described polypeptides can be synthesized using conventional synthesis procedures commonly used by one skilled in the art. For example, the polypeptides can be chemically synthesized using an automated peptide synthesizer (such as one manufactured by Pharmacia LKB Biotechnology Co., LKB Biolynk 4170 or Milligen, Model 9050 (Milligen, Millford, Mass.)) following the method of Sheppard, et al., Journal of Chemical Society Perkin I, p. 538 (1981). In this procedure, N,N′-dicyclohexylcarbodiimide is added to amino acids whose amine functional groups are protected by 9-flourenylmethoxycarbonyl (Fmoc) groups and anhydrides of the desired amino acids are produced. These Fmoc-amino acid anhydrides can then be used for peptide synthesis. A Fmoc-amino acid anhydride corresponding to the C-terminal amino acid residue is fixed to Ultrosyn A resin through the carboxyl group using dimethylaminopyridine as a catalyst. Next, the resin is washed with dimethylformamide containing piperidine, and the protecting group of the amino functional group of the C-terminal acid is removed. The next amino acid corresponding to the desired peptide is coupled to the C-terminal amino acid. The deprotecting process is then repeated. Successive desired amino acids are fixed in the same manner until the peptide chain of the desired sequence is formed. The protective groups other than the acetoamidomethyl are then removed and the peptide is released with solvent.

Alternatively, the polypeptides can be synthesized by using nucleic acid molecules which encode the polypeptides of this invention in an appropriate expression vector which include the encoding nucleotide sequences. Such DNA molecules may be prepared using an automated DNA sequencer and the well-known codon-amino acid relationship of the genetic code. Such a DNA molecule also may be obtained as genomic DNA or as cDNA using oligonucleotide probes and conventional hybridization methodologies. Such DNA molecules may be incorporated into expression vectors, including plasmids, which are adapted for the expression of the DNA and production of the polypeptide in a suitable host such as bacterium, e.g., Escherichia coli, yeast cell, mammalian cell, or insect cell. Mammalian expression systems may facilitate glycosylation that may improve pharmaceutical and/or immunologic properties of the compound.

Another aspect of the invention is to provide fragments, analogs, and derivatives of the polypeptides of the present invention. The terms “fragment,” “derivative” and “analog” as used herein refer to compounds modified in such manner as to retain the ability to bind RANK. Thus, a fragment may be any suitable portion of the peptide of the present invention so long as the RANK binding functionality is retained by the fragment. Modifications may be achieved by any of the techniques known in the art for derivatization of polypeptides into fragments, analogs, or derivatives thereof. Such terms and in particular, “analog”, also specifically include peptide, non-peptide, small molecules and other compounds that function as RANKL mimics that bind RANKL.

Those of ordinary skill in the art are aware that modifications in the amino acid sequence of a peptide, polypeptide, or protein can result in equivalent, or possibly improved, second generation peptides, etc., that display equivalent or superior functional characteristics when compared to the original amino acid sequence. The present invention accordingly encompasses such modified amino acid sequences. Alterations can include but are not limited to amino acid insertions, deletions, substitutions, truncations, fusions, cyclization, disulfide bridging, shuffling of subunit sequences, and the like, provided that the peptide sequences produced by such modifications retain the ability to bind RANK. Such modifications may be undertaken to improve compound half-life, biological activity, absorption, distribution, metabolism, excretion, toxicity or the like. One factor that can be considered in making such changes is the hydropathic index of amino acids. The importance of the hydropathic amino acid index in conferring interactive biological function on a protein has been discussed by Kyte and Doolittle (J. Mol. Biol., 157: 105-132, 1982). It is accepted that the relative hydropathic character of amino acids contributes to the secondary structure of the resultant protein. This, in turn, affects the interaction of the protein with molecules such as enzymes, substrates, receptors, DNA, antibodies, antigens, etc.

Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide or protein can be substituted for other amino acids having a similar hydropathic index or score and produce a resultant peptide or protein having similar biological activity, i.e., which still retains biological functionality. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant protein having similar biological activity, i.e., still retaining correct biological function. In making such changes, amino acids having hydropathic indices within ±2 are preferably substituted for one another, those within ±1 are more preferred, and those within ±0.5 are most preferred.

As outlined above, amino acid substitutions in the polypeptides of the present invention can be based on the relative similarity of the amino acid side-chain substituents, for example, their hydrophobicity, hydrophilicity, charge, size, etc. Exemplary substitutions that take various of the foregoing characteristics into consideration in order to produce conservative amino acid changes resulting in silent changes within the present polypeptides, etc., can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine. It should be noted that changes which are not expected to be advantageous can also be useful if these result in the production of functional sequences.

Thus, the fragment, derivative or analog of the polypeptides of the present invention may be, for example and without limitation, (i) one in which one or more amino acid residues are substituted with a conserved or non-conserved amino acid residue, and such substituted amino acid residue may or may not be one encoded by the genetic code; (ii) one in which one or more of the amino acid residues includes a substituent group; (iii) one in which the mature protein is fused to another compound such as a compound to increase the half-life of the protein; (iv) one in which additional amino acids are fused to the protein to aid in purification or in detection and identification; or (v) one in which additional amino acid residues are fused to the protein to aid in modifying tissue distribution or localization of the protein to certain locations such as the cell membrane or extracellular compartments; or (vi) one in which another molecule, possibly a small, non-peptide molecule, mimics the RANK-binding functionality of the polypeptide.

It is standard practice in the art to modify polypeptides to improve their potential as pharmacological agents. Thus, modifications can be made without completely affecting a polypeptide's binding to RANK and/or its ability to inhibit osteoclast differentiation. For example, oligomerization of RANKL has been shown to be a useful technique for delaying internalization of the protein. In addition, cyclization may be used to stabilize the RANKL mimics of the present invention. Similarly, known treatments to increase the stability or other beneficial characteristic of the polypeptide, such as substitution of L- with D-amino acids, or PEG-alation, may be utilized to like effect with the RANKL mimics of the present invention.

In one aspect, the polypeptide sequence may be flanked at either of both of its N- and/or C-termini by residues. When included, such flanking residues should not significantly alter the ability of the core sequence to bind to RANK and inhibit RANK/RANKL interaction. Flanking residues may include Cysteines to facilitate disulfide bridging. Thus, in an embodiment, the polypeptides of the invention may include flanking residues at each terminus that may be fewer than 5 residues each. In a preferred embodiment, fewer than 3 flanking residues at each terminus, and most preferably no flanking residues.

Such molecules may contain a number of modifications known to those skilled in the art. For instance, substituted amide linkages may generally include, but are not limited to, groups of the formula —C(O)N(R)—, where R is (C₁-C₆) alkyl, substituted (C₁-C₆) alkyl, (C₂-C₆) alkenyl, substituted (C₂-C₆) alkenyl, (C₂-C₆) alkynyl, substituted (C₂-C₆) alkynyl, (C₅-C₂₀) aryl, substituted (C₅-C₂₀) aryl, (C₆-C₂₆) arylalkyl, substituted (C₆-C₂₆) arylalkyl, 5-20 membered heteroaryl, substituted 5-20 membered heteroaryl, 6-26 membered heteroarylalkyl and substituted 6-26 membered heteroarylalkyl.

Isosteres of amide linkages may generally include, but are not limited to, —CH₂NH—, —CH₂S—, —CH₂CH₂—, —CH═CH— (cis and trans), —C(O)CH₂—, —CH(OH)CH₂— and —CH₂SO—. Compounds having such non-amide linkages and methods for preparing such compounds are well-known in the art (see, e.g., Spatola, March 1983, Vega Data Vol. 1, Issue 3; Spatola, 1983, “Peptide Backbone Modifications” In: Chemistry and Biochemistry of Amino Acids Peptides and Proteins, Weinstein, ed., Marcel Dekker, New York, p. 267 (general review); Morley, 1980, Trends Pharm. Sci. 1:463-468; Hudson et al., 1979, Int. J. Prot. Res. 14:177-185 (—CH₂NH—, —CH₂CH₂—); Spatola et al., 1986, Life Sci. 38:1243-1249 (—CH₂—S); Hann, 1982, J. Chem. Soc. Perkin Trans. I. 1:307-314 (—CH═CH—, cis and trans); Almquist et al., 1980, J. Med. Chem. 23:1392-1398 (—COCH₂—); Jennings-White et al., Tetrahedron. Lett. 23:2533 (—COCH₂—); European Patent Application EP 45665 (1982) CA 97:39405 (—CH(OH)CH₂—); Holladay et al., 1983, Tetrahedron Lett. 24:4401-4404 (—C(OH)CH₂—); and Hruby, 1982, Life Sci. 31:189-199 (—CH₂—S—).

Additionally, one or more amide linkages can be replaced with peptidomimetic or amide mimetic moieties which do not significantly interfere with the structure or activity of the peptides. Alternatively, all of amide linkages may be replaced with peptidomimetic moieties. Suitable amide mimetic moieties are described, for example, in Olson et al., 1993, J. Med. Chem. 36:3039-3049.

The peptides and peptide analogs may optionally include a peptide or peptide analog at either or both termini that may be 1 to 5 residues or more in length. Peptide analogs typically contain at least one modified interlinkage, such as a substituted amide or an isostere of an amide, as described above. Such additional peptides or peptide analogs may have an amino acid sequence derived from another portion of the RANKL amino acid sequence or, alternatively, their sequences may be completely random. Peptides including such random sequences may be tested for biological activity, i.e. their ability to bind to RANK.

One method of testing compound binding to RANK is determined by performing an assay such as, e.g., a binding assay between a desired compound and RANK. In one aspect, this is done by contacting said compound to RANK and determining its dissociation rate. Numerous possibilities for performing binding assays are well known in the art. The indication of a compound's ability to bind to RANK is determined, e.g., by a dissociation rate, and the correlation of binding activity and dissociation rates is well established in the art.

For example, the assay may be performed by radio-labeling a reference compound, peptide, or protein such as RANKL or isolated external surface loops therefrom, e.g. with ¹²⁵l and incubating it with RANK in 1.5 ml tubes. Test compound s are then added to these reactions in increasing concentrations. After optimal incubation, the RANK/compound complexes are separated, e.g., with chromatography columns, and evaluated for bound ¹²⁵l-labeled peptide with γ counter. The amount of the test compound necessary to inhibit 50% of the reference peptide's binding is determined. These values are then normalized to the concentration of unlabeled reference peptide's binding (relative inhibitory concentration (RIC)⁻¹=concentrationₜₑₛₜ/concentration_{reference}). A small RIC⁻¹ value indicates strong relative binding, whereas a large RIC⁻¹ value indicates weak relative binding. See, for example, Latek et al., Proc. Natl. Acad. Sci. USA, Vol. 97, No. 21, pp. 11460-11465, 2000. Of course, high throughput binding assays such as those offered commercially by PerkinElmer, Actelion and others, are also suitable for testing compound binding.

Also included within the scope of the present invention are “blocked” forms of the peptides and peptide analogs in which the N- and/or C-terminus is blocked with a moiety capable of reacting with the N-terminal —NH₂ or C-terminal —C(O)OH. Such blocked compounds are typically N-terminal acylated and/or C-terminal amidated or esterified. Typical N-terminal blocking groups include R¹C(O)—, where R¹ is hydrogen, (C₁-C₆) alkyl, (C₂-C₆) alkenyl, (C₂-C₆) alkynyl, (C₅-C₂₀) aryl, (C₆-C₂₆) arylalkyl, 5-20 membered heteroaryl or 6-26 membered heteroarylalkyl. Preferred N-terminal blocking groups include acetyl, formyl and dansyl. Typical C-terminal blocking groups include —C(O)NR¹R¹ and —C(O)OR¹, where each R¹ is independently as defined above. Preferred C-terminal blocking groups include those in which each R¹ is independently (C₁-C₆) alkyl, preferably methyl, ethyl, propyl or isopropyl.

In a preferred embodiment of the invention, a method of preventing or inhibiting bone loss, a method of inhibiting osteoclast differentiation, and a method of competitively inhibiting RANKL activity are provided by administering compositions comprising compounds identified by the screening methods described herein. The bone forming compositions of the present invention may be utilized by providing an effective amount of such compositions to a subject in need thereof. The methods and compositions may be used to treat many diseases or conditions characterized by bone loss or thinning. Such diseases and conditions include osteoporosis, juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, corticosteroid treatment, metastatic bone diseases, periodontal bone loss, bone loss due to cancer, age-related loss of bone mass, and other forms of osteopenia. In a preferred embodiment, the methods and compositions of the invention are used to treat osteoporosis, osteolytic bone disease, bone loss due to rheumatoid arthritis, and skeletal metastasis.

For use for treatment of animal subjects, the compositions of the invention can be formulated as pharmaceutical or veterinary compositions. Depending on the subject to be treated, the mode of administration, and the type of treatment desired, e.g., prevention, prophylaxis, therapy; the compositions are formulated in ways consonant with these parameters. A summary of such techniques is found in Remington's Pharmaceutical Sciences, latest edition, Mack Publishing Co., Easton, Pa.

The administration of the compositions of the present invention may be pharmacokinetically and pharmacodynamically controlled by calibrating various parameters of administration, including the frequency, dosage, duration mode and route of administration. Variations in the dosage, duration and mode of administration may also be manipulated to produce the activity required.

For administration to animal or human subjects, the dosage of the compounds of the invention is typically 0.01-100 mg/kg. However, dosage levels are highly dependent on the nature of the disease or situation, the condition of the patient, the judgment of the practitioner, and the frequency and mode of administration. If the oral route is employed, the absorption of the substance will be a factor effecting bioavailability. A low absorption will have the effect that in the gastro-intestinal tract higher concentrations, and thus higher dosages, will be necessary.

It will be understood that the appropriate dosage of the substance should suitably be assessed by performing animal model tests, wherein the effective dose level (e.g. ED₅₀) and the toxic dose level (e.g. TD₅₀) as well as the lethal dose level (e.g. LD₅₀ or LD₁₀) are established in suitable and acceptable animal models. Further, if a substance has proven efficient in such animal tests, controlled clinical trials should be performed.

In general, for use in treatment, the compounds of the invention may be used alone or in combination with other compositions for the treatment of bone loss. Such compositions include anti-resorptives such as a bisphosphonate, a calcitonin, a calcitriol, an estrogen, SERM's and a calcium source, or a supplemental bone formation agent like parathyroid hormone or its derivative, a bone morphogenetic protein, osteogenin, NaF, or a statin. See U.S. Pat. No. 6,080,779 incorporated herein by reference. Depending on the mode of administration, the compounds will be formulated into suitable compositions.

Formulations may be prepared in a manner suitable for systemic administration or for topical or local administration. Systemic formulations include, but are not limited to those designed for injection (e.g., intramuscular, intravenous or subcutaneous injection) or may be prepared for transdermal, transmucosal, nasal, or oral administration. The formulation will generally include a diluent as well as, in some cases, adjuvants, buffers, preservatives and the like.

For oral administration, the compositions can be administered also in liposomal compositions or as microemulsions. Suitable forms include syrups, capsules, tablets, as is understood in the art. For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

The compositions of the present invention may also be administered locally to sites in patients, both human and other vertebrates, such as domestic animals, rodents and livestock, where decreased bone loss and/or increased bone mass are desired using a variety of techniques known to those skilled in the art. For example, these may include sprays, lotions, gels or other vehicles such as alcohols, polyglycols, esters, oils and silicones. Such local applications include, for example, at a site of a bone fracture or defect to repair or replace damaged bone. Additionally, an anti-resorptive agent may be administered e.g., in a suitable carrier, at a junction of an autograft, allograft or prosthesis and native bone to assist in binding of the graft or prosthesis to the native bone.

Another embodiment of the present invention involves use of the RANKL loops in competitive binding assays to screen for inhibitors of RANKL. Binding to RANK is determined by performing an assay as described above as a binding assay between a desired compound and RANK. In one aspect, this is done by contacting a test compound to RANK and determining its dissociation rate. Numerous possibilities for performing binding assays are well known in the art. The indication of a compound's ability to bind to RANK is determined, e.g., by a dissociation rate, and the correlation of binding activity and dissociation rates is well established in the art. For example, the assay may be performed by radio-labeling a reference compound, e.g. a polypeptide containing a portion of AA″ loop sequence SEQ ID NO 1 with ¹²⁵I and incubating it with RANK in 1.5 ml tubes. Test compounds are then added to these reactions in increasing concentrations. After optimal incubation, the RANK/compound complexes are separated, e.g., with chromatography columns, and evaluated for bound ¹²⁵I-labeled peptide with gamma (γ) counter. The amount of the test compound necessary to inhibit 50% of the reference compound's binding is determined. These values are then normalized to the concentration of unlabeled reference compound's binding (relative inhibitory concentration (RIC)⁻¹=concentrationₜₑₛₜ/concentration_{reference}). A small RIC⁻¹ value indicates strong relative binding, whereas a large RIC⁻¹ value indicates weak relative binding. See, for example, Latek et al., Proc. Natl. Acad. Sci. USA, Vol. 97, No. 21, pp. 11460-11465,2000. The RANKL loops identified in SEQ ID NO 2-5 and fragments thereof are also suitable for use individually or in combination as reference compounds in such assays. Again, high throughput assays are also suitable to perform the binding assays involving one or more of the RANKL loops.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

All publications and patent applications cited in this specification are herein incorporated by reference as if each individual publication or patent application was specifically and individually indicated to be incorporated by reference.

The following examples illustrate the invention, but are not to be taken as limiting the various aspects of the invention so illustrated.

### EXAMPLES

### Example 1

The effect of a partial AA″ loop polypeptide of RANKL on TRAP activity. Wild type C3H/HENJ mice were purchased from Harlan Industries (Indianapolis, Ind.). In order to establish a cell culture of osteoclast precursors, bone marrow macrophages (BMMs) were isolated from whole bone marrow of four to six week old mice and incubated in tissue culture dishes at 37° C. in 5% CO₂. After 24 hours in culture, the non-adherent cells were collected and layered on a Ficoll Hypaque gradient and the cells at the gradient interface were collected. Cells were replated at 65,000/cm² in α-minimal essential medium, supplemented with 10% heat inactivated fetal bovine serum, at 37° C. in 5% CO₂ in the presence of recombinant mouse M-CSF (10 ng.ml). The cell cultures were then treated either with the increasing concentrations of polypeptide of SEQ ID NO 1 (subset of AA″ loop) or with a negative control scrambled peptide having the same molecular weight as the test compound.

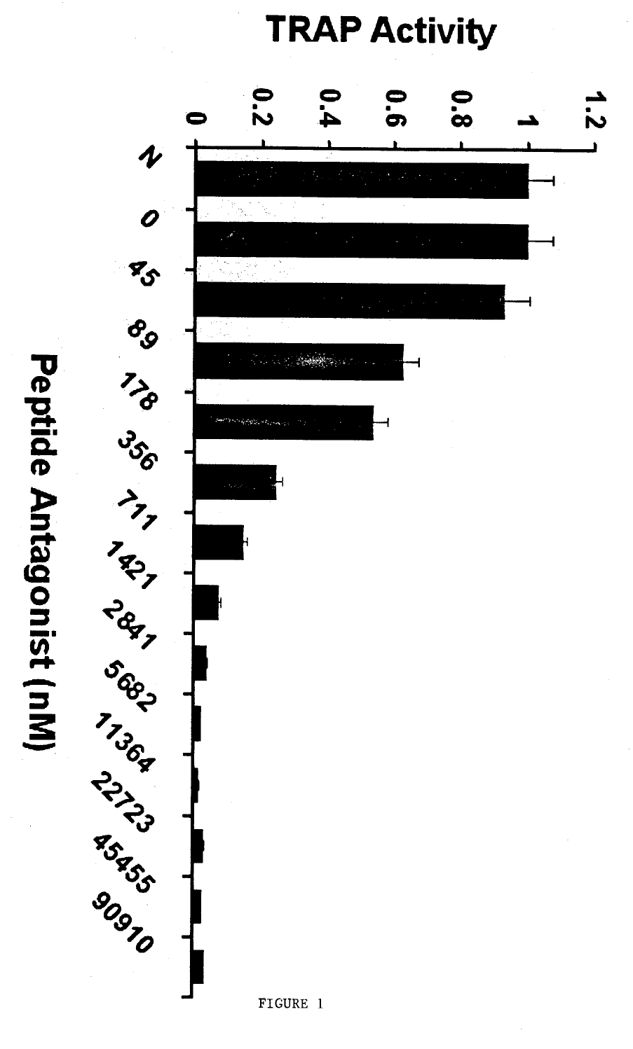

The results of the experiment are shown in FIG. 1. As can be seen from the same figure, addition of the test compound to the cell cultures of osteoclast precursors competitively inhibits the ability of RANKL to induce osteoclast differentiation as monitored by tartrate specific acid phosphatase (TRAP). This phosphatase is an osteoclast specific enzyme and its activity corresponds with osteoclast differentiation. TRAP activity is measured by quantitating its ability to cleave a substrate in a color-producing reaction.

As can also be seen from FIG. 1, the osteoclast differentiation inhibition is dose-dependent, i.e. increasing the concentration of the test compound in culture decreases TRAP activity, whereas the negative control peptide has no affect on TRAP activity.

What is claimed is:

1. An isolated polypeptide comprising one or more amino acid sequences selected from SEQ ID NO 1-SEQ ID NO 5 and SEQ ID NO: 7-SEQ ID NO: 11, said polypeptide having the ability to bind RANK.

2. A polypeptide of claim 1 having conservative or non-conservative modifications and still having the ability to bind to RANK.

3. A polypeptide comprising a RANKL sequence involved in binding to RANK, wherein said sequence consists essentially of one or more amino acid sequences selected from SEQ ID NO 1-SEQ ID NO 5 and SEQ ID NO: 7-SEQ ID NO: 11, and said polypeptide has the ability to bind to RANK.

4. The polypeptide of claim 3 having conservative or non-conservative modifications and having the ability to inhibit RANKL binding with RANK.

5. A polypeptide, other than RANKL, comprising one or more external surface loops of RANKL, said polypeptide having the ability to bind to RANK.

6. A polypeptide comprising one or more external surface loops of RANKL, said polypeptide having the ability to competitively inhibit RANKL binding with RANK.

7. The polypeptide of claim 1 consisting essentially of the amino acid sequence selected from SEQ ID NO 1-SEQ ID NO 5 and SEQ ID NO: 7-SEQ ID NO: 11.

8. A compound comprising a fragment, analog, mimic or derivative of the polypeptide of claim 1, 2, 3, 4, 5, 6, or 7, said compound having the ability to bind to RANK.

9. A pharmaceutical composition comprising an effective amount of the polypeptide of claim 1, 2, 3, 4, 5, 6, 7, in a pharmaceutically acceptable carrier, adjuvant, solubilizer, stabilizer, and/or anti-oxidant.

10. A pharmaceutical composition comprising an effective amount of the compound of claim 8 in a pharmaceutically acceptable carrier, adjuvant, solubilizer, stabilizer, and/or anti-oxidant.

11. A method of inhibiting osteoclast differentiation comprising administering an effective amount of the pharmaceutical composition of claim 9.

12. A method of inhibiting bone resorption comprising administering an effective amount of the pharmaceutical composition of claim 9.

13. A method of competitively inhibiting RANKL, said method comprising administering an effective amount of the compound of claim 1-8.

14. A method of competitively inhibiting RANKL, said method comprising administering an effective amount of the pharmaceutical composition of claim 9.

15. A method of treating diseases or conditions selected from the group comprising osteoporosis, juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, corticosteroid treatment, metastatic bone diseases, periodontal bone loss, bone loss due to cancer, age-related loss of bone mass, and other forms of osteopenia, wherein the method comprises administering an effective amount of the pharmaceutical composition of claim 9.

16. The method of claim 15, wherein the diseases or conditions comprise osteoporosis, osteolytic bone disease, rheumatoid arthritis, and skeletal metastasis.

17. A polypeptide comprising (a) a RANKL sequence involved in binding to RANK, wherein said sequence consists essentially of at least one amino acid sequence selected from SEQ ID NO 1-SEQ ID NO 5 and SEQ ID NO: 7-SEQ ID NO: 11, and said polypeptide has the ability to bind to RANK; and (b) additional amino acid residues flanking said sequence that do not eliminate the ability of the sequence to bind RANK.

18. The polypeptide of claim 1 having one or more of the following modifications:
(i) one in which one or more amino acid residues of the polypeptide are substituted with a conserved or non-conserved amino acid residue, such residue may or may not be one encoded by the genetic code;
(ii) one in which one or more of the amino acid residues includes a substituent group;
(iii) one in which the polypeptide is fused to another compound such as a compound to increase the half-life of the protein;
(iv) one in which additional amino acids are fused to the polypeptide to aid in purification or in detection and identification; or
(v) one in which additional amino acid residues are fused to the polypeptide to aid in modifying tissue distribution or localization of the protein to certain locations such as the cell membrane or extracellular compartments.

19. A method of screening for RANKL-binding compounds, said method comprising performing a binding assay between a test compound, a reference compound and RANK wherein said reference compound consists essentially of a polypeptide of at least one amino acid sequence selected from SEQ ID NO 1-SEQ ID NO 5 and SEQ ID NO: 7-SEQ ID NO: 11.

20. A polynucleotide encoding the polypeptide of claim 1.

21. A polynucleotide of claim 20 which exhibit regular degeneracy in accordance with the degeneracy of the genetic code.

22. A polynucleotide of claim 20 encoding a polypeptide exhibiting conservative substitutions to the polypeptide of claim 1.

23. An expression vector comprising the polynucleotide of claim 20, 21, or 22.

24. A host cell containing an expression vector of claim 23.

25. The method of treating diseases of claim 15 further comprising administering an effective amount of one or more pharmaceutical compositions for enhancing bone growth to a subject in need thereof.
